(19) United States
(12) Patent Application Publication
(10) Pub. No.: US 2014/0378336 A1
(43) Pub. Date: Dec. 25, 2014

(54) DIAGNOSIS OF CELL PROLIFERATIVE DISEASES

(71) Applicant: NEW YORK UNIVERSITY, New York, NY (US)

(72) Inventors: Harvey I. Pass, Bronxville, NY (US); Chandra Goparaju, Cliffside Park, NJ (US)

(73) Assignee: NEW YORK UNIVERSITY, New York, NY (US)

(21) Appl. No.: 14/366,721

(22) PCT Filed: Dec. 21, 2012
(86) PCT No.: PCT/US2012/071111

§ 371 Date: Jun. 19, 2014

Related U.S. Application Data
(60) Provisional application No. 61/578,748, filed on Dec. 21, 2011.
(60) Provisional application No. 61/712,607, filed on Oct. 11, 2012.

(51) Int. Cl. C12Q 1/68; G01N 33/574

(52) U.S. Cl. CPC C12Q 1/6886; G01N 33/57488
USPC 506/9; 435/6.12; 435/6.11; 435/7.92; 435/7.1

(57) ABSTRACT

The method of the present disclosure is directed towards a method and a kit for diagnosing a cell proliferative disease. This method includes the following steps; gathering a sample from a subject, measuring the fibulin expression level or measuring the fibulin activity level in the sample, comparing the measured fibulin expression level or measured fibulin activity level to a baseline level of fibulin expression or activity and assigning the subject to a normal or abnormal group based on the measured fibulin expression level or activity level in the sample. The present disclosure is also directed towards a kit for diagnosing a cell proliferative disease. The kit includes a sampler, a measurer, a comparor and an assignor.

### CROSS REFERENCE TO RELATED APPLICATIONS

This application claims priority from U.S. Provisional Application No. 61/712,607, filed Oct. 11, 2012 and U.S. Provisional Application No. 61/578,748, filed Dec. 21, 2011, which are herein incorporated by reference in their entireties.

### STATEMENT REGARDING FEDERALLY SPONSORED RESEARCH OR DEVELOPMENT

This disclosure was sponsored by an agency of the United States Government, specifically, the National Cancer Institute, under government contract number 2U01CA 111295-04. The government has rights in this disclosure.

### FIELD OF THE DISCLOSURE

The disclosure relates generally to the field of diagnosing cell proliferative diseases. More specifically, the present disclosure is directed to measuring levels of fibulin for diagnosing cell proliferative diseases in a patient.

### BACKGROUND OF THE DISCLOSURE

Fibulins are a family of extracellular matrix proteins, comprising a family of widely expressed extracellular matrix (ECM) proteins. There are several identified members of the fibulin family, which are defined by the presence of two structural modules, a tandem repeat of epidermal growth factor-like modules and a C-terminal fibulin-type module. Fibulins mediate cell-to-cell and cell-to-matrix communication, as well as provide organization and stabilization to EXM structures during organogenesis and vasculogenesis. A component of physiologic tissue development and repair is angiogenesis, which when left unchecked can promote cell proliferative diseases such as cancer.

### SUMMARY OF THE DISCLOSURE

The present disclosure is directed to a method for diagnosing a cell proliferative disease including the steps of; gathering a sample from a subject, measuring the fibulin expression level or measuring the fibulin activity level in the sample, comparing the measured fibulin expression level or measured fibulin activity level to a baseline level of fibulin expression or activity and assigning the subject to a normal or abnormal group based on the measured fibulin expression level or activity level in the sample.

The present disclosure is also directed to a kit for diagnosing a cell proliferative disease, the kit including; a sampler, the sampler configured to withdraw a sample from a subject, a measurer, the measure configured to measure the fibulin expression level or measuring the fibulin activity level in the sample, a comparor, the comparor configured to compare the measured fibulin expression level or measured fibulin activity level to a baseline level of fibulin expression or activity and an assignor, the assignor configured to assign the subject to a normal or abnormal group based on the measured fibulin expression level or activity level in the sample.

Embodiments of the present application provide a method and kit that include the above.

### BRIEF DESCRIPTION OF THE DRAWINGS

The present disclosure will be better understood by reference to the following drawings of which:

FIG. 1 is a graphical representation of the values of fibulin between subjects who have been exposed to asbestos and those with mesothelioma.

FIG. 2 is a graphical representation of the values of fibulin between subjects who have been exposed to asbestos and subjects in stage I or stage II mesothelioma.

FIG. 3 is a graphical representation of the values of fibulin between subjects with non-MSM effusions and those with mesothelioma.

FIG. 4 is a graphical representation of the values of fibulin between subjects with benign effusions and those with mesothelioma.

FIG. 5 is a graphical representation of the values of fibulin between subjects with non-mesothelioma cancer and those with mesothelioma.

FIG. 6 is a graphical representation comparing effusion fibulin levels of cytroreduced patients.

FIG. 7 is a graphical representation of the survival of cytoreduced patients over time.

### DETAILED DESCRIPTION OF THE DISCLOSURE

### Definitions

The term “fibulin” is meant to describe the members of the multigene family of fibulins, which are secreted glycoproteins.

The term “fibulin expression” is meant to describe transcription and/or translation of the fibulin protein in a quasi cell environment. The term “fibulin expression” can also mean the detection of fibulin translation. This approach lends itself to specific protein labeling with fluorescence, biotin, radioactivity or heavy atoms, via modified charged tRNA's or amino acids. Methods suitable for the detection of fibulin protein include any suitable method for detecting and/or measuring proteins from a cell or cell extract. Such methods are known in the art. Furthermore, antibodies against specific fibulins are known in the art.

The term “fibulin activity” is meant to describe any biological action of the protein.

The method of the present disclosure provides a method, and a kit to practice the method, to diagnose cell proliferative diseases. The cell proliferative disease can be any cell proliferative disease, including but not limited to mesothelioma.

The present disclosure is directed towards a method for diagnosing a cell proliferative disease comprising several steps. Initially, a sample is gathered from a subject. In one embodiment this subject is a human subject. The sample can be any material capable of fibulin expression or activity measurement, including but not limited to the subject's plasma, the subject's serum, the subject's pleural or peritoneal effusion and tumorigenic tissue of the subject. The sample can be gathered by any suitable apparatus, including but not limited to a hypodermic needle.

Once the sample is gathered, the fibulin expression level or the fibulin activity level is measured. The fibulin expression level or fibulin activity level can be those of any fibulin, including but not limited to Fibulin-3.

Fibulin-3 is one of the several fibulins and is shown as SEQ ID NO: 1. Fibulin-3 is encoded by the EFEMP1 gene is located at chromosome 2p16, contains 11 exons encoding a 493-amino acid protein with a molecular mass of 54 kD. The long form of Fibulin-3 comprises the 1-493 amino acids of SEQ ID NO: 1, while the short form of Fibulin-3 comprises amino acids 107-493 of SEQ ID NO: 1.

The fibulin expression level or fibulin activity level can be measured in several different ways, including but not limited to detecting fibulin mRNA transcription, detecting fibulin protein and detecting fibulin biological activity.

When the measuring step includes detecting fibulin mRNA transcription expression by cells in the sample, the detection can be done with any suitable method, including but not limited to a polymerase chain reaction (PCR), a reverse transcriptase-PCR (RT-PCR), an in situ hybridization, a Northern blot, a sequence analysis, a gene microarray analysis and the detection of a reporter gene.

When the measuring step includes detecting fibulin protein expression in the sample, the detection can be done with any suitable method, including but not limited to an immunoblot, an enzyme-linked immunosorbant assay (ELISA), a radioimmunoassay (RIA), an immunoprecipitation, an immunohistochemistry and an immunofluorescence.

When the measuring step includes detecting fibulin biological activity in the sample, the detection can be done with any suitable method, including but not limited to measuring proliferation of cells expressing fibulin, detecting DNA synthesis in cells expressing fibulin, detecting MAP kinase activity in cells expressing fibulin, detecting MAP kinase activity in the sample, measuring migration and migration and invasion ability of fibroblasts expressing fibulin, detecting the ability of fibulin to regulate vascular endothelial growth factor (VEGF) signaling, detecting the ability of fibulin to regulate matrix metalloproteinase (MMP) expression and activity and detecting the ability of fibulin to regulate tissue inhibitor of metalloproteinase (TIMP) expression.

Once the fibulin expression level or fibulin activity level is measured, the measured value is compared to a baseline level of fibulin expression or activity. This comparison can be done by anything capable of comparing values, such as a human operator or a suitably programmed computer hardware and/or software.

Once the fibulin expression level or fibulin activity level of the subject is measured and compared to a baseline level, the subject is assigned to a normal or abnormal group based on the comparison. The subject is assigned to an abnormal group if the measured fibulin expression level or fibulin activity level is above the baseline level. Conversely, the subject is assigned to a normal group if the measured fibulin expression level or fibulin activity level is below the baseline level.

In one embodiment, in the assignment step, the normal group comprises a group of patients exposed to asbestos that do not have mesothelioma and the abnormal group comprises a group of patients with mesothelioma. When the baseline of fibulin expression or fibulin activity level is about 52 ng/mL, the assigning step is about 95% specific in assigning between the normal group and the abnormal group and about 95% sensitive in assigning between the normal group and the abnormal group.

In another embodiment, in the assignment step, the normal group comprises a group of patients with non-mesothelioma benign and malignant pleural effusions and the abnormal group comprises a group of patients with mesothelioma. When the baseline of fibulin expression or fibulin activity level is about 67 ng/mL, the assigning step is about 93% specific in assigning between the normal group and the abnormal group and about 78% sensitive in assigning between the normal group and the abnormal group.

In another embodiment, in the assignment step, the normal group comprises a group of patients exposed to asbestos that do not have mesothelioma and the abnormal group comprises a group of patients with non-mesothelioma benign and malignant pleural effusions. When the baseline of fibulin expression or fibulin activity level is about 34 ng/mL, the assigning step is about 82% specific in assigning between the normal group and the abnormal group and about 73% sensitive in assigning between the normal group and the abnormal group.

The disclosure also includes a kit that can be used for diagnosing a cell proliferative disease. The kit includes several components, including but not limited to a sampler, a measurer, a comparor and an assignor.

The sampler can be any component configured to withdraw a sample from a subject, such as a hypodermic needle. The measurer can be any component that is capable of measuring fibulin expression level or measuring fibulin activity level in the sample withdrawn by the sampler. Suitable measurers can be any measurer, including those disclosed herein, that can detect the fibulin expression level or fibulin activity level. These suitable measurers can detect fibulin mRNA transcription by cells in the sample, detect fibulin protein in the sample or detect fibulin biological activity in the sample. The comparor can be any component that is capable of comparing the measured fibulin expression or fibulin activity level in the sample and compare the measured fibulin expression or fibulin activity level to a baseline level of fibulin expression or activity. This comparor can be any component that is capable of doing this comparison including but not limited to a sufficiently programmed computer hardware and/or software or a human operator. The kit also includes an assignor component. The assignor can be any component that is capable of assigning the subject to a normal or abnormal group based on the measured fibulin expression level or activity level in the sample. This assignor can be any assignor capable of doing this assignment, including but not limited to a sufficiently programmed computer hardware and/or software or a human user.

### Example 1

Confirmation of the methods described herein was conducted with samples from human subjects over a period of several years.

Three groups of subjects were studied from two separate institutions, Wayne State University (WSU) and New York University (NYU). The group of subjects includes 41 from WSU and 95 from NYU with a history of asbestos exposure, evidence of asbestosis, or both (AE); 53 from WSU and 40 from NYU with benign or non-mesothelioma malignant effusions; and 82 from WSU (37 with plasma) and 64 from NYU (55 with plasma) with pleural mesothelioma (MSM). All subjects provided plasma and/or pleural effusion.

Of the 41 subjects from the WSU AE cohort, 41 had plasma available, 32 (78%) had fiber exposure in their professions for at least five years, 5 (12%) had exposure of less than five years, while 4 (10%) had radiographic evidence of abnormalities consistent with exposure to asbestos despite reporting only passive exposure. Radiographic evidence of fibrosis was found in 13 (33%) and pleural scarring and plaques were found in 30 (75%).

Of the 95 subjects from the NYU AE cohort, four subjects (4%) did not relate an occupational exposure to asbestos, the other 91 had exposures of greater than five years (6-58 years) with 60 (63%) having pleural scarring, 23 (24%) having plaques and four (5%) having parenchymal changes.

Of the 82 WSU subjects with MSM, Ethylenediaminetetraacetic acid (EDTA) samples were obtained from 37 subjects with histologically proven MSM. In addition to these plasma samples, pleural effusion aliquots were obtained for 53, for which 8 had matching plasma samples. Exposure to asbestos by occupation or passive means was recorded in 66 (80%) of the subjects. All subjects were cytoreduced or were staged clinically and radiographically during surgical procedures, undergoing a complete pathologic surgical staging according to the staging system of the International Mesotheilioma Interest Group (IMIG).

Of the 64 NYU subjects with MSM, EDTA plasma was obtained from 55 subjects with histologically proven MSM. Pleural effusion aliquots were obtained from 21 subjects, for which 12 had matching plasma samples. Exposure to asbestos either by occupation or passive means was documented in 45 (70) subjects. All subjects were cytoreduced or were staged clinically and radiographically during surgical procedures, undergoing a complete pathologic surgical staging according to the staging system of IMIG.

Of the 53 WSU subjects with benign or non-MPM malignant effusions, the group included the following histologically or cytologically confirmed (28 malignant, 25 benign) fluids: asbestos inflammatory effusion (1), chronic inflammation (2), congestive heart failure (1), hydropneumothorax (2), past immune/chemotherapy (2), postoperative effusion (17), adenocarcinoma of the lung (12), squamous carcinoma of the lung (4), lung cancer not otherwise specified (8), renal cell cancer (2), breast cancer (1) and lymphoma (1).

Of the 40 NYU subjects with benign or non-MPM malignant effusions, the group included the following histologically or cytologically confirmed (26 malignant, 14 benign) fluids: asbestos inflammatory effusion (2), chronic inflammation (4), congestive heart failure (1), reactive pleural effusion (7), adenocarcinoma of the lung (7), squamous carcinoma of the lung (2), lung cancer not otherwise specified (3), adenocarcinoma gastrointestinal (3), renal cell cancer 91), sarcoma (1), cancer unknown primary (1), breast cancer (5), small cell carcinoma (2) and lymphoma (1).

Fibulin-3 enzyme was measured with a linked immunosorbent assay, available from USCN Life Science Inc., to determine the level of plasma and pleural effusion Fibulin-3. Each specimen was tested in duplicate, the results quantified in nanograms per milliliter using a standard curve.

To determine how Fibulin-3 levels distinguish subjects with MSM from AE subjects and non-MSM effusions, Fibulin-3 was evaluated by descriptive statistics and receiver-operating-characteristic (ROC) curves. The area under the ROC (AUC) was calculated, 95% confidence intervals were used to compare to the theoretical AUC of 0.5. An AUC with a confidence interval that did not include the 0.5 value was considered to be evidence that the laboratory test did not have an ability to distinguish between the two groups. The differences between the groups was calculated by using analysis of variance and multiple regression analysis in a stepwise fashion, entering variables with a p value of less than 0.05 in the model. All statistical analyses were performed using MedCalc software.

Cutoff values for detection were determined for both pleural effusion samples and for plasma samples. The cutoff values for plasma samples will be described first.

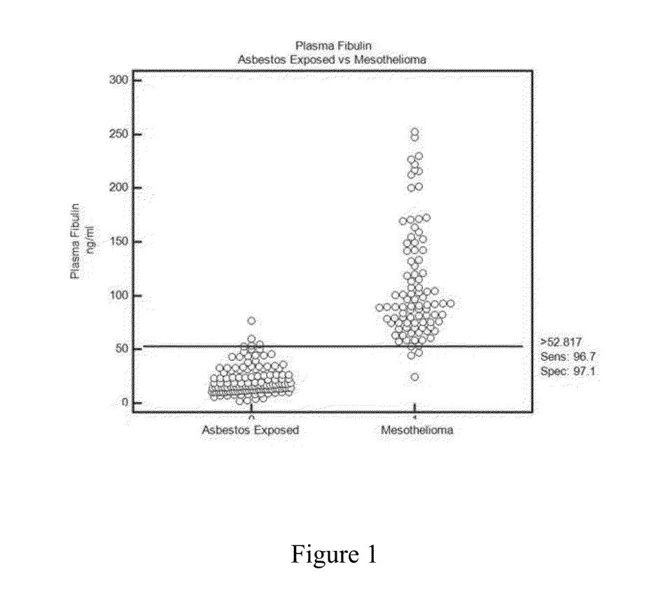

In order to determine the specificity of fibulin for the detection of MSM, levels of Fibulin-3 present in MPM subjects was compared to subjects with non-MPM effusions and AE subjects. For the detection of MSM in the subjects as compared to AE subjects, plasma fibulin levels discriminated MSM from AE. The cut-off for maximum sensitivity and specificity for the analysis was about 52 ng/mL, with sensitivity being ˜95%, specificity being ˜95% and an AUC of 0.99. When specificity was set to 100%, sensitivity was ˜67% at a cut-off value of about 76 ng/mL. When sensitivity was set at 100%, the specificity was ˜65% at a cut-off value of about 24 ng/mL The results for the NYU and WSU subjects are shown in FIG. 1.

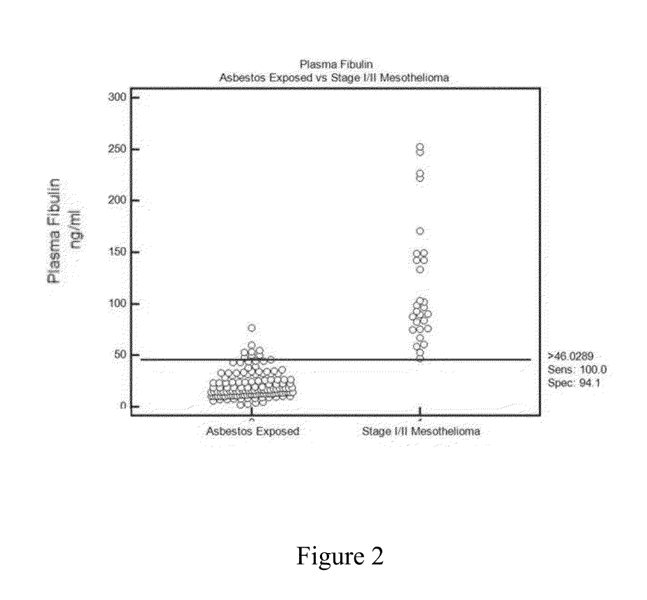

The measurement of Fibulin-3 levels can also be used to compare AE subjects with subjects in stage I or stage II MSM. For the analysis of assigning a subject's sample to AE or stage I/II MSM, the cut-off for maximum sensitivity and specificity for the analysis was about 46 ng/mL, with sensitivity being ˜99% and specificity being ˜94%. The results for the NYU and WSU subjects are shown in FIG. 2.

For the detection of non-MPM effusions in the subjects as compared to AE, plasma fibulin levels discriminated non-MPM from AE. The cut-off for maximum sensitivity and specificity was about 34 ng/mL, with sensitivity being ˜73%, specificity being ˜82% and an AUC of 0.81. When specificity was set to 100%, sensitivity was ˜3% at a cut-off value of about 76 ng/mL. When sensitivity was set to 100%, specificity was 0% at a cut-off value of ˜6 ng/mL.

For the detection of MPM in the subjects as compared to non-MPM effusions, plasma fibulin levels discriminated MPM from non-MPM effusions. The cut-off for maximum sensitivity and specificity was about 67 ng/mL, with a sensitivity of ˜78%, a specificity of ˜93% and an AUC of 0.92. When specificity was set to 100%, sensitivity was 49% at a cut-off value of about 90 ng/mL When sensitivity was set to 100%, specificity was ˜17% at a cut-off value of about 24 ng/mL.

The cutoff values for pleural effusion samples are described in the following paragraphs.

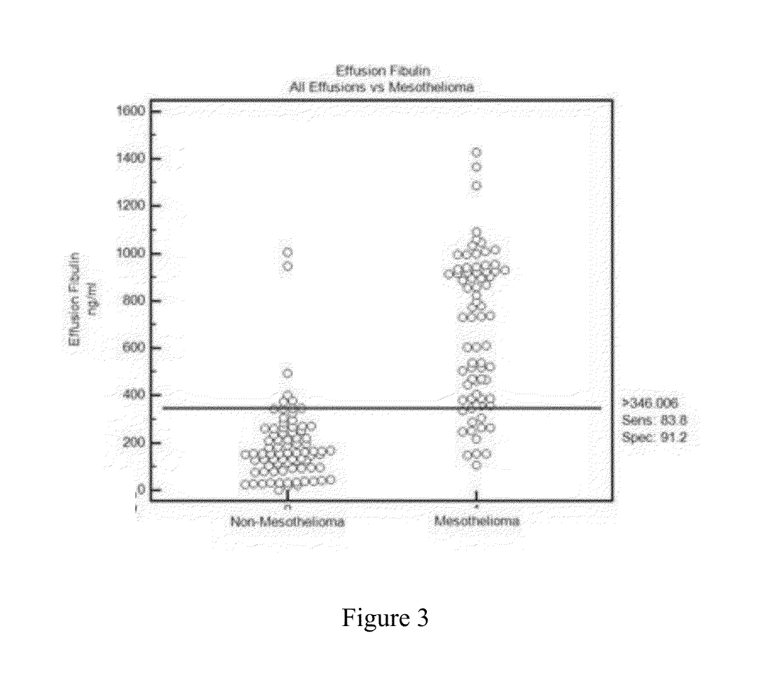

In order to determine the specificity of fibulin for the detection of MSM, levels of Fibulin-3 present in MPM subjects was compared to subjects with any non-MPM effusions. For the detection of MSM in the subjects as compared to all non-MPM effusions, pleural effusion fibulin levels discriminated MSM from all non-MPM effusions. The cut-off for maximum sensitivity and specificity was about 346 ng/mL, with sensitivity being ˜84%, specificity being ˜91% and an AUC of 0.92. When specificity was set at 100%, the sensitivity was ˜28% at a cut-off value of about 750 ng/mL. When sensitivity was set to 100%, the specificity was ˜44% at a cut-off value of about 114 ng/mL. The results for the NYU and WSU subjects are shown in FIG. 3.

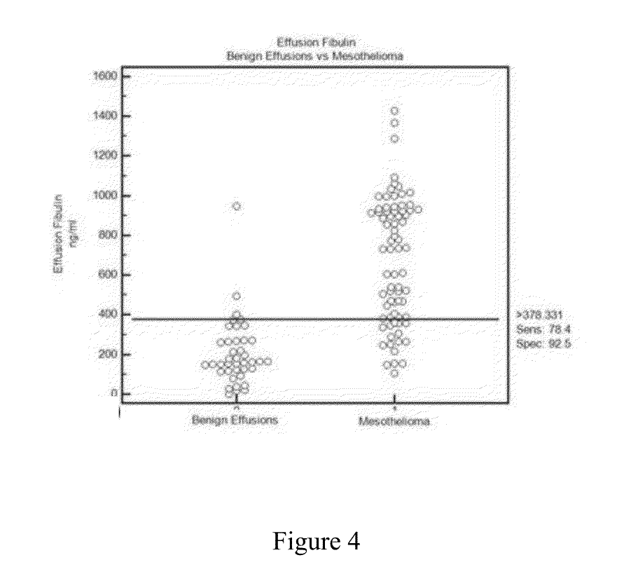

Further, levels of Fibulin-3 present in MSM subjects was compared to subjects with benign effusions. The cut-off for maximum sensitivity and specificity was about 378 ng/mL, with sensitivity being ˜78% and specificity being ˜93%. The results for the NYU and WSU subjects are shown in FIG. 4.

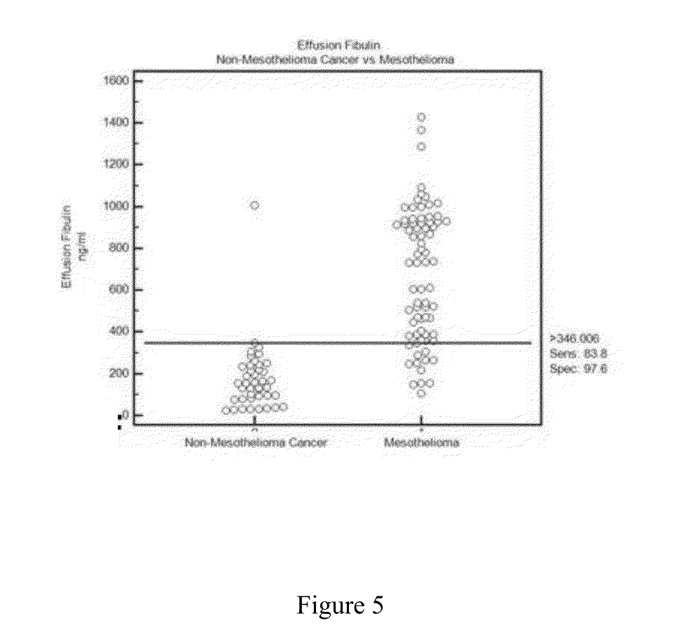

Further still, levels of Fibulin-3 present in MSM subjects was compared to subjects with non-MSM cancers. The cut-off for maximum sensitivity and specificity was about 346 ng/mL, with sensitivity being ˜84% and specificity being 98%. The results for the NYU and WSU subjects are shown in FIG. 5.

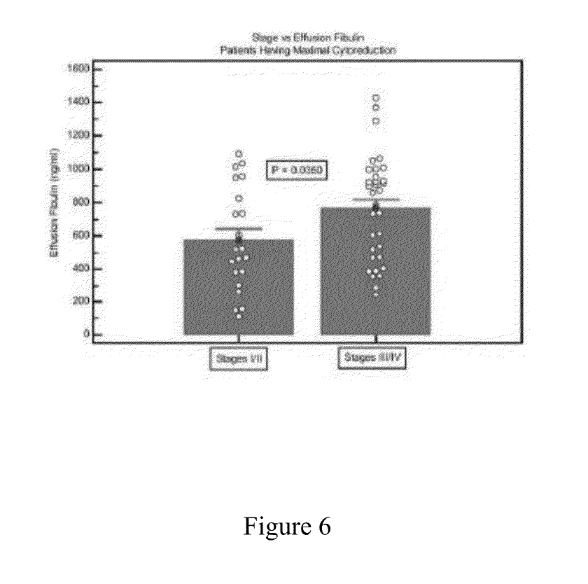

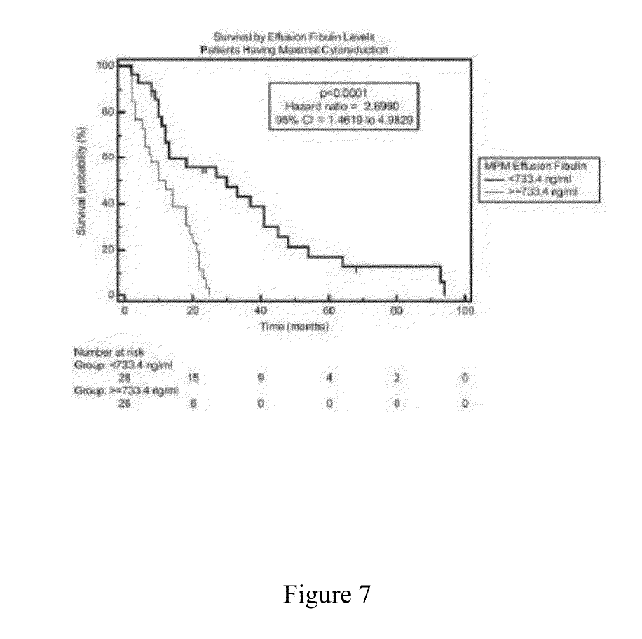

Effusion fibulin levels in 54 pathologically staged, cytoreduced patients revelaed significant differences in 21 Stage I/II patients compared to 33 Stage III/IV patients, as shown in FIG. 6. Moreover, using the median effusion fibulin level of 733.4 ng/mL of all 69 MPMs with survival information as a cut-off, a significant difference in survival was noticed in the cytoreduced patients based on their effusion fibulin levels at the time of surgery, as illustrated in FIG. 7.

To determine serum vs plasma fibulin, twenty samples (10 from WSU, 10 from NYU) having matched serum and plasma aliquots were used. The correlation coefficient was 0.94 with a confidence index (CI) of 0.835-0.9767. The levels of plasma fibulin was not significantly different when comparing quartiles of ages of either r the asbestos exposed individuals or the subjects with MSM. For the subjects with MSM, there was little difference in the levels of plasma fibulin when comparing males to females or comparing epithelial histology to biphasic or sarcomatoid.

### Example 2

A walk through example will now be described. This walk through example provides one example of a subject being assigned to a normal or abnormal group.

Initially, a person goes to a facility where the several steps of the method are able to be performed or a facility that includes the described kit. Once the person is at the facility, their serum is gathered with a hypodermic needle and the fibulin expression level is measured using an ELISA.

The person was concerned whether they had developed MSM since they had been exposed to asbestos previously. Since the person was concerned about whether or not they have MSM, the measured fibulin expression level is compared to a baseline of about 52 ng/mL. Since the person's fibulin expression level is below 52 ng/mL, they are assigned to the normal group, meaning that they do not have MSM even though they were exposed to asbestos.

This assignment is 95% specific and correctly assigns 95% of people that have been exposed to asbestos but do not have mesothelioma to a normal group.

1. A method for diagnosing a cell proliferative disease comprising the steps of:
gathering a sample from a subject;
measuring the fibulin expression level or measuring the fibulin activity level in the sample;
comparing the measured fibulin expression level or measured fibulin activity level to a baseline level of fibulin expression or activity; and
assigning the subject to a normal or abnormal group based on the measured fibulin expression level or activity level in the sample.

2. The method of claim 1, wherein the cell proliferative disease is mesothelioma.

3. The method of claim 1, wherein the normal group comprises a group of patients exposed to asbestos that do not have mesothelioma and the abnormal group comprises a group of patients with mesothelioma.

4. The method of claim 3, wherein the assigning step is about 95% specific between the normal group and the abnormal group and about 95% sensitive between the normal group and the abnormal group.

5. The method of claim 1, wherein the normal group comprises a group of patients with non-mesothelioma benign and malignant pleural effusions and the abnormal group comprises a group of patients with mesothelioma.

6. The method of claim 5, wherein the assigning step is about 93% specific between the normal group and the abnormal group and about 78% sensitive between the normal group and the abnormal group.

7. The method of claim 1, wherein the normal group comprises a group of patients exposed to asbestos that do not have mesothelioma and the abnormal group comprises a group of patients with non-mesothelioma benign and malignant pleural effusions.

8. The method of claim 7, wherein the assigning step is about 82% specific between the normal group and the abnormal group and about 73% sensitive between the normal group and abnormal group.

9. The method of claim 1, wherein the sample is selected from the group consisting of the subject's plasma, the subject's serum, the subject's pleural or peritoneal effusion and the subject's tumorigenic tissue.

10. The method of claim 1, wherein the subject is human.

11. The method of claim 1, wherein the fibulin is fibulin-3.

12. The method of claim 1, wherein the measuring step comprises detecting fibulin mRNA transcription by cells in the sample.

13. The method of claim 12, wherein the fibulin concentration is measured by a method selected from the group consisting of polymerase chain reaction (PCR), reverse transcriptase-PCR (RT-PCR), in situ hybridization, Northern blot, sequence analysis, gene microarray analysis and detection of a reporter gene.

14. The method of claim 1, wherein the measuring step comprises detecting fibulin protein in the sample.

15. The method of claim 14, wherein the step of measuring is by a method selected from the group consisting of immunoblot, enzyme-linked immunosorbant assay (ELISA), radioimmunoassay (RIA), immunoprecipitation, immunohistochemistry and immunefluorescence.

16. The method of claim 1, wherein the measuring step comprises detecting fibulin biological activity in the sample.

17. The method of claim 16, wherein the measuring step comprises measuring proliferation of cells expressing fibulin, detecting DNA synthesis in cells expressing fibulin, detecting MAP kinase activity in cells expressing fibulin, detecting MAP kinase activity in the sample, and measuring migration and migration and invasion ability of fibroblasts expressing fibulin, detecting the ability of fibulin to regulate vascular endothelial growth factor (VEGF) signaling, detecting the ability of fibulin to regulate matrix metalloproteinase (MMP) expression and activity and detecting the ability of fibulin to regulate tissue inhibitor of metalloproteinase (TIMP) expression.

18. A kit for diagnosing a cell proliferative disease, the kit comprising:
a sampler, the sampler configured to withdraw a sample from a subject;
a measurer, the measure configured to measure the fibulin expression level or measuring the fibulin activity level in the sample;
a comparor, the comparor configured to compare the measured fibulin expression level or measured fibulin activity level to a baseline level of fibulin expression or activity; and
an assignor, the assignor configured to assign the subject to a normal or abnormal group based on the measured fibulin expression level or activity level in the sample.

19. The kit of claim 18, wherein the measurer comprises detecting fibulin mRNA transcription by cells in the sample, detecting fibulin protein in the sample or detecting fibulin biological activity in the sample.


SEQUENCE LISTING (1 sequence)

SEQ ID NO: 1 - protein, 493 aa, Artificial Sequence
  Fibulin-3
MLKALFLTMLTLALVKSQDTEETITYTQCTDGYEWDPVRQQCKDIDECDIVPDACKGGMKCVNHYGGYLCLPKTAQIIVNNEQPQQETQPAEGTSGATTGVVAASSMATSGVLPGGGFVASAAAVAGPEMQTGRNNPVIRRNPADPQRIPSNPSHRIQCAAGYEQSEHNVCQDIDECTAGTHNCRADQVCINLRGSFACQCPPGYQKRGEQCVDIDECTIPPYCHQRCVNTPGSFYCQCSPGFQLAANNYTCVDINECDASNQCAQQCYNILGSFICQCNQGYELSSDRLNCEDIDECRTSSYLCQYQCVNEPGKFSCMCPQGYQVVRSRTCQDINECQTTNECREDEMCWNYHGGFRCYPRNPCQDPYILTPENRCVCPVSNAMCRELPQSIVYKYMSIRSDRSVPSDIFQIQATTIYANTINTFRIKSGNENEEFYLRQTSPVSAMLVLVKSLSGPREHIVDLEMLTVSSIGTFRTSSVLRLTIIVGPFSF